(19) United States
(12) Patent Application Publication
(10) Pub. No.: US 2025/0360151 A1
(43) Pub. Date: Nov. 27, 2025

(54) NUTRITIONAL COMPOSITION COMPRISING GOS AND HMOS

(71) Applicant: SOCIETE DES PRODUITS NESTLE S.A., Vevey (CH)

(72) Inventors: LAURENT FERRIER, Servion (CH); GABRIELA BERGONZELLI DEGONDA, Bussigny (CH); IVANA JANKOVIC, Mex (CH)

(21) Appl. No.: 18/872,906

(22) PCT Filed: May 16, 2023
(86) PCT No.: PCT/EP2023/063176

§ 371 Date: Dec. 9, 2024

(30) Foreign Application Priority Data
Jun. 9, 2022 (EP) 22178116.4

(51) Int. Cl. A61K 31/702; A61P 25/22; A61P 25/24

(52) U.S. Cl. CPC A61K 31/702; A61P 25/22; A61P 25/24

(57) ABSTRACT

The present invention relates to a combination of galacto-oligosaccharides (GOS) and a mixture of human milk oligosaccharides (HMOs), or nutritional compositions comprising the combination. The combination or compositions are for use in treating and/or preventing stress and/or a mood disorder in an infant, young child and/or child. The mixture of HMOs consists or consists essentially of 2′-fucosyllactose (2′FL) and difucosyllactose (DFL) or of lacto-N-tetraose (LNT) and 6′ sialyllactose (6′SL). The present invention also relates to the use of a combination of galacto-oligosaccharides (GOS) and a mixture of human milk oligosaccharides (HMOs), or compositions comprising the combination, for treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

### FIELD OF THE INVENTION

The present invention relates to a combination of galacto-oligosaccharides (GOS) and a mixture of human milk oligosaccharides (HMOs), or compositions comprising the combination, particularly for use in treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL). The present invention also relates to the use of a combination of galacto-oligosaccharides (GOS) and a mixture of human milk oligosaccharides (HMOs), or compositions comprising the combination, for treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

### BACKGROUND OF THE INVENTION

Infants and children are often exposed to psychological stress such as changes in social situation (e.g., changes in care provider or schools, new family additions, medical conditions or diseases). When the stress is extreme, extended exposure may lead to counter-adaptive physiological and behavioural responses such as: anxiety or distress, heightened sensitivity to painful stimuli, depression, and impaired neuronal development or neuronal functioning. Pharmaceutical interventions may not be appropriate for use in infants and children due to potential side effects on neuronal development. There is a need for nutritional solutions (i.e. non-pharmaceutical solutions) that treat or prevent stress.

Gamma-aminobutyric acid (GABA) is the chief inhibitory neurotransmitter. It is present in the central nervous system (CNS), but is also present in high amounts in foods like crustaceans, plants and is naturally produced by certain bacteria. Therefore, GABA is naturally present in the gut.

Several gut bacteria have been shown to synthesize GABA (Wiley et al., 2021, Mod. Trends Psychiatry, 32:74-79). There is clinical and preclinical evidence of the effect of exogenous GABA in relieving stress and promoting the sensation of feeling relaxed (Abdou et al., 2006, BioFactors, 26:201-208). Probiotics producing GABA have been shown to alleviate gut discomfort triggered by stress in preclinical studies (Pokusaeva et al., 2016, Neurogastroenterol. Motil., 29: e12904).

There is no nutritional solution shown or available to naturally increase the synthesis of GABA by the microbiota of an infant, young child and/or child exposed to the nutritional solution, in particular in treating and/or preventing stress and/or a mood disorder, for example in maintaining or restoring the functionality of the bi-directional transmission pathways in the gut-brain axis to healthy levels.

Accordingly, there is a need to provide nutritional solutions capable of treating and/or preventing stress and/or a mood disorder, for example in maintaining or restoring the functionality of the bi-directional transmission pathways in the gut-brain axis to healthy levels.

### SUMMARY OF THE INVENTION

The present inventors surprisingly found that novel combinations of galacto-oligosaccharide (GOS) and a mixture of human milk oligosaccharides (HMOs) can advantageously be used in treating and/or preventing stress and/or a mood disorder because the combination showed increased biosynthesis of gamma-aminobutyric acid (GABA) by the gut microbiota of the infant, young child and/or child. The present inventors have found that these specific combinations are particularly effective in increasing biosynthesis of gamma-aminobutyric acid (GABA) by the gut microbiota of the infant, young child and/or child. Thus, the invention provides a way to increase the production of a metabolite (i.e. GABA) with known benefits on stress relief and relaxation. The mixture of HMOs consists or consists essentially of either 2′-FL and DFL or of LNT and 6′-SL. The impact of a combination of GOS and a mixture of HMOs on the biosysnthesis of GABA by microbiota was not previously known. The combination is particularly effective in the restoration of the sensitivity of the bi-directional transmission pathways in the gut-brain axis to normal levels.

In the experiment at the basis of the present invention, a combination of GOS and either 2′-FL and DFL or LNT and 6′-SL resulted in increased biosynthesis of GABA by microbiota. Thus, the experiment indicates the role played by the combination in promoting the biosynthesis of GABA.

Thus, in a first aspect, the present invention provides a combination of galacto-oligosaccharides (GOS) and a mixture of human milk oligosaccharides (HMOs), wherein the mixture of HMOs consists or consists essentially of 2′-fucosyllactose (2′FL) and difucosyllactose (DFL) or of lacto-N-tetraose (LNT) and 6′ sialyllactose (6′SL).

In another aspect, the present invention provides nutritional composition comprising a combination of galacto-oligosaccharides (GOS) and a mixture of human milk oligosaccharides (HMOs), wherein the mixture of HMOs consists or consists essentially of 2′-fucosyllactose (2′FL) and difucosyllactose (DFL) or of lacto-N-tetraose (LNT) and 6′ sialyllactose (6′SL). The nutritional composition is particularly for an infant formula, a starter infant formula, a follow-on or follow-up infant formula, a growing-up milk, a baby food, an infant cereal composition, a fortifier suitable for an infant, young child and/or child or a supplement suitable for an infant, young child and/or child.

In another aspect, the present invention provides a combination of galacto-oligosaccharides (GOS) and a mixture of human milk oligosaccharides (HMOs) for use in treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL). Suitably, the combination treats stress and/or a mood disorder by increasing production of gamma-aminobutyric acid (GABA) by the gut microbiota of the infant, young child and/or child.

In another aspect, the invention provides a combination of galacto-oligosaccharides (GOS) and a mixture of human milk oligosaccharides (HMOs) for use in maintaining or restoring the functionality of the bi-directional transmission pathways in the gut-brain axis to healthy levels in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In another aspect, the invention provides a combination of galacto-oligosaccharides (GOS) and a mixture of human milk oligosaccharides (HMOs) for use in increasing production of gamma-aminobutyric acid (GABA) by the gut microbiota of an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In another aspect, the present invention provides a nutritional composition comprising a combination of galacto-oligosaccharides (GOS) and a mixture of human milk oligosaccharides (HMOs) for use in treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL). Suitably, the composition treats stress and/or a mood disorder by increasing production of gamma-aminobutyric acid (GABA) by the gut microbiota of the infant, young child and/or child.

In another aspect, the invention provides a nutritional composition comprising a combination of galacto-oligosaccharides (GOS) and a mixture of human milk oligosaccharides (HMOs) for use in maintaining or restoring the functionality of the bi-directional transmission pathways in the gut-brain axis to healthy levels in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In another aspect, the invention provides a nutritional composition comprising a combination of galacto-oligosaccharides (GOS) and a mixture of human milk oligosaccharides (HMOs) for use in increasing production of gamma-aminobutyric acid (GABA) by the gut microbiota of an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In a further aspect, the present invention provides a method for treating and/or preventing stress and/or a mood disorder in an infant, young child and/or child, the method comprising administering to an infant, young child and/or child a combination according to the invention or a nutritional composition according to the invention.

In a further aspect, the present invention provides a method for maintaining or restoring the functionality of the bi-directional transmission pathways in the gut-brain axis to healthy levels in an infant, young child and/or child, the method comprising administering to an infant, young child and/or child a combination according to the invention or a nutritional composition according to the invention.

In a further aspect, the present invention provides a method for increasing production of gamma-aminobutyric acid (GABA) by the gut microbiota of an infant, young child and/or child, the method comprising administering to an infant, young child and/or child a combination according to the invention or a nutritional composition according to the invention.

In a further aspect, the present invention provides the use of a combination according to the invention or a nutritional composition according to the invention for treating and/or preventing stress and/or a mood disorder in an infant, young child and/or child. Suitably, the combination treats stress and/or a mood disorder by increasing production of gamma-aminobutyric acid (GABA) by the gut microbiota of the infant, young child and/or child.

In another aspect, the invention provides the use of a combination according to the invention or a nutritional composition according to the invention for maintaining or restoring the functionality of the bi-directional transmission pathways in the gut-brain axis to healthy levels in an infant, young child and/or child.

In another aspect, the invention provides the use of a combination according to the invention or a nutritional composition according to the invention for increasing production of gamma-aminobutyric acid (GABA) by the gut microbiota of an infant, young child and/or child.

### BRIEF DESCRIPTION OF THE FIGURES

FIG. 1: Relative abundance of GABA, expressed as peak area, after 24 h of fermentation by toddler's microbiota of digested milk matrix supplemented by GOS, 2′FL/DFL, LNT/6′SL, GOS+2′FL/DFL or GOS+LNT/6′SL. Dots represent individual values, line is at the median, box are 25/75 percentiles and whiskers at min/max range. Different letters denote significant differences.

FIG. 2: Relative abundance of GABA, expressed as the base-2 logarithm of the ratio between the value of GABA after 24 h of fermentation by toddler's microbiota of digested milk matrix supplemented by GOS, 2′FL/DFL, LNT/6′SL, GOS+2′FL/DFL or GOS+LNT/6′SL, and the value of GABA obtained after fermentation of the digested milk matrix alone. Dots represent individual values, line is at the median, box are 25/75 percentiles and whiskers at min/max range. Different letters denote significant differences.

### DETAILED DESCRIPTION OF THE INVENTION

### Definitions

The term “infant” means a child under the age of 12 months. The expression “young child” means a child aged between one and less than three years, also called toddler. The expression “child” means a child between three and seven years of age. Preferably, the expression “child” means a child between three and five years of age.

An “infant, young child or child born by C-section” means an infant, young child or child who was delivered by caesarean. It means that the infant, young child or child was not vaginally delivered.

An “infant, young child or child vaginally born” means an infant, young child or child who was vaginally delivered and not delivered by caesarean.

A “preterm” or “premature” means an infant, young child or child who was not born at term. Generally it refers to an infant, young child or child born prior 37 weeks of gestation.

An “infant having a low birth weight” means a new born having a body weight below 2500 g (5.5 pounds) either because of preterm birth or restricted fetal growth. It therefore encompasses:
infant, young child or child who has/had a body weight from 1500 to 2500 g at birth (usually called “low birth weight” or LBW)
infant, young child or child who has/had a body weight from 1000 to 1500 g at birth (called “very low birth weight” or VLBW)
infant, young child or child who has/had a body weight under 1000 g at birth (called “extremely low birth weight” or ELBW).

An “infant born small for gestational age (SGA)” means a baby with birth weights below the 10th percentile for babies of the same gestational age. It therefore encompasses an infant, young child or child who has/had a birth weight below the 10th percentile for babies of the same gestational age.

The expression “nutritional composition” means a composition which nourishes a subject. This nutritional composition is usually to be taken orally or parenterally, and it usually includes a lipid or fat source and a protein source. A carbohydrate source may also be included. In one embodiment, the nutritional composition of the invention is a synthetic nutritional composition.

In a particular embodiment, the combination or composition of the present invention is a “synthetic combination” or “synthetic nutritional composition”. The expression “synthetic combination” or “synthetic nutritional composition” means a mixture obtained by chemical and/or biological means, which can be chemically identical to the mixture naturally occurring in mammalian milks (i.e. the synthetic combination or synthetic composition is not breast milk).

The expression “infant formula” as used herein refers to a foodstuff intended for particular nutritional use by infants during the first months of life and satisfying by itself the nutritional requirements of this category of person (Article 2 (c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The term “fortifier” refers to liquid or solid nutritional compositions suitable for fortifying or mixing with human milk, infant formula, growing-up milk or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The expression “weaning period” means the period during which the mother's milk is substituted by other food in the diet of an infant or young child.

The expressions “days/weeks/months/years of life” and “days/weeks/months/years after birth” can be used interchangeably.

The “mother's milk” should be understood as the breast milk or the colostrum of the mother.

An “oligosaccharide” is a saccharide polymer containing a small number (typically three to ten) of simple sugars (monosaccharides).

The term “galacto-oligosaccharides” refers to a type of non-digestible fiber with prebiotic activity. GOS are formed via enzymatic conversion of lactose. GOS generally comprise a chain of galactose units that arise through consecutive transgalactosylation reactions, with a terminal glucose unit, although a terminal galactose unit may be present instead. The degree of polymerization of GOS typically ranges from 2 to 8 monomeric units. Such GOS contain β-(1→2), β-(1→3), β-(1→4), or β-(1→6) linked galactose moieties and may have a degree of polymerization of 3 to 8 galactose units. The term GOS is therefore preferably referred to as oligosaccharide(s) comprising at least three galactose units, more preferably as oligosaccharide(s) comprising at least four galactose units, preferably having a degree of polymerization of 3 to 8 galactose units.

The term “HMO” or “HMOs” refers to human milk oligosaccharide(s). These carbohydrates are highly resistant to enzymatic hydrolysis, indicating that they may display essential functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk-over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide).

A “fucosylated oligosaccharide” is an oligosaccharide having a fucose residue. It has a neutral nature. Some examples are 2′FL (2′-fucosyllactose), 3-FL (3-fucosyllactose), difucosyllactose (DFL), Lacto-difucotetraose (LDFT)), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

A “precursor of HMO” is a key compound that intervenes in the manufacture of HMO, such as sialic acid and/or fucose.

A “sialylated oligosaccharide” is a charged sialic acid containing oligosaccharide, i.e. an oligosaccharide having a sialic acid residue. It has an acidic nature. Some examples are 3-SL (3′ sialyllactose) and 6′SL (6′ sialyllactose).

The combination or nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or a growing-up milk or an infant cereal product or any other formulation designed for infant nutrition).

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

“Gamma-aminobutyric acid” or “GABA” is commonly known as an inhibitory neurotransmitter in the central nervous system and for its function to inhibit neuronal activity involved, for example, in synchronizing network of neurons in the hippocampus.

“Treating” means to address a medical condition or disease with the objective of improving or stabilising an outcome in the person being treated or addressing an underlying nutritional need. Treating, therefore, includes the dietary or nutritional management of the medical condition or disease by addressing nutritional needs of the person being treated. Treating includes the elimination, reduction or amelioration of symptoms associated with the medical condition or disease.

“Preventing” means to diminish the risk of onset or recurrence of a medical condition or disease. Both primary and secondary prevention are thus contemplated. “Primary prevention” means preventing a medical condition or disease before it occurs, and “secondary prevention” means preventing additional attacks of a medical condition or disease after the first attack has occurred. Preventing, therefore, includes the dietary or nutritional prophylaxis of the medical condition or disease by addressing nutritional needs of the person being treated. Preventing includes eliminating or minimising the risk of developing a medical condition or disease and reducing the risk of developing symptoms associated with the medical condition or disease.

The term “stress” means a state of emotional or psychological strain. Stress may be caused by adverse or demanding circumstances.

The term “stress resilience” means the ability to adapt successfully when faced with stress. Hence, the individual may avoid symptoms associated with stress.

The term “mood” refers to a positive or negative emotional state of varying intensity that changes in response to life's circumstances (Polak et al., (2015) Nutrition for brain health and cognitive performance, 95-122). Mood, like emotion, is an affective state or ‘a feeling’ and has different aspects such as alertness/fatigue, anxiety, and stress. Moods can be distinguished from emotions in terms of duration and the (in) ability to describe the trigger. Moods are often undifferentiated, slow to change, can last from days to weeks, and are ‘object-less’ in that people may not know the cause or the source of the mood (J. A. Russell, (2003, Psychological review, 110:145-172). Emotions have clear focus (i.e. a self-evident cause) and are short in duration, i.e. seconds to minutes. Moods and emotions are closely linked, it is common for bad moods to be accompanied by negative emotions, and good moods to accompany positive emotions.

The term “mood disorder” refers to a category of illnesses that describe a significant and serious change in mood. Mood disorders include: anxiety disorder, depression, bipolar disorder and SAD (seasonal affective disorder). In some embodiments, the mood disorder is anxiety disorder or depression. In some embodiments, the mood disorder is anxiety. In some embodiments, the mood disorder is depression.

The term “anxiety” refers to a feeling of unease, such as worry, nervousness, or fear, that can be mild or severe, particularly about something which is about to happen or may happen in the future.

The term “anxiety disorder” refers to a condition of intense, excessive and persistent worry and fear about everyday situations. The symptoms can interfere with daily activities such as schoolwork and relationships. There are several types of anxiety disorders, including generalized anxiety disorder, separation anxiety disorder, panic disorder, social anxiety disorder, and various phobia-related disorders.

The term “depression” (also known as major depressive disorder) refers to a condition of persistently low mood (feeling sad, irritable, empty or loss of pleasure/interest). The symptoms can interfere with daily activities such as schoolwork and relationships.

The term “bipolar disorder” means a condition that causes extreme mood swings that include emotional highs (mania) and lows (depression). Mania can be characterised by euphoria, hyperactivity, over inflated ego and unrealistic optimism.

“Promoting relaxation” means enhancing the sensation of being relaxed, e.g. increasing calmness.

“Increasing production of gamma-aminobutyric acid (GABA) by the gut microbiota of an infant, young child and/or child” means enhancing the biosynthesis of GABA by the gut microbiota of an infant, young child or child administered the combination or nutritional composition of the invention compared to the biosynthesis of GABA by the gut microbiota of an infant, young child or child not administered the combination or nutritional composition of the invention.

“Maintaining the functionality of the bi-directional transmission pathways in the gut-brain axis to healthy levels” means maintaining the bi-directional transmission pathways in the gut-brain axis of an infant, young child or child at normal levels, i.e. those of a healthy infant, young child or child.

“Restoring the functionality of the bi-directional transmission pathways in the gut-brain axis to healthy levels” means altering the bi-directional transmission pathways in the gut-brain axis of an infant, young child or child suffering from a medical condition, disease or dysregulation of the transmission pathways in the gut-brain axis to normal levels, i.e. to those of a healthy infant, young child or child.

The term “effective amount” preferably means an amount of the combination or composition that provides the combination of GOS and the mixture of HMOs in a sufficient amount to render a desired treatment or prevention outcome in a subject. An effective amount can be administered in one or more doses to the subject to achieve the desired treatment or prevention outcome.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs. “Consist(s) essentially” of given element(s) or “consisting essentially” of given element(s) means that the element(s) are comprised in an amount of 95% to 100% by weight, preferably in an amount of 97% to 100% by weight, more preferably in an amount of 99% to 100% by weight, most preferably in an amount of 99.5% to 100% by weight, such as in an amount of 99.9% to 100% by weight.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

### Use

In one aspect the present invention provides a combination of galacto-oligosaccharides (GOS) and a mixture of human milk oligosaccharides (HMOs) for use in treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

The combination may be in the form of a nutritional composition.

Accordingly, in another aspect, the present invention provides a nutritional composition comprising a combination of galacto-oligosaccharides (GOS) and a mixture of human milk oligosaccharides (HMOs) for use in treating and/or preventing stress and/or a mood disorder in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

There is clinical and preclinical evidence of the effect of exogenous GABA in relieving stress and promoting the sensation of feeling relaxed (Abdou et al., 2006, BioFactors, 26:201-208). Probiotics producing GABA have been shown to alleviate gut discomfort triggered by stress in preclinical studies (Pokusaeva et al., 2016, Neurogastroenterol. Motil., 29: e12904). The effect of oral GABA administration on stress is reviewed by Hepsomali et al. (Hepsomali et al., (2020), Front. Neurosci., 14:923). GABA has been shown to reduce salivary cortisol in humans (Kanehira T et al., (2011), J Nutr Sci Vitaminol (Tokyo), 57:9-15). However, no nutritional solution has previously been shown to naturally increase the synthesis of GABA by the microbiota of an infants, and/or young child and/or child exposed to the nutritional solution.

Treating includes the elimination, reduction or amelioration of symptoms associated with the medical condition or disease.

In some embodiments, treating improves the stress resilience of an infant, young child and/or child.

Symptoms of stress that may be eliminated, reduced or ameliorated include anxiety, distress, depression, heightened sensitivity to painful stimuli, and impaired neuronal development or neuronal functioning. Suitably, the treatment may reduce the amount of cortisol in the saliva, which is a measure of stress (Jessop D S, Turner-Cobb J M, (2008), Stress, 11:1-14; Bi et al., (2020), J Anim Physiol Anim Nutr (Berl)., 104:590-596; Kanehira T et al., (2011), J Nutr Sci Vitaminol (Tokyo), 57:9-15).

In some embodiments, the mood disorder is anxiety disorder or depression.

Symptoms of anxiety disorder that may be eliminated, reduced or ameliorated include finding it hard to concentrate, difficulty sleeping, irritability, crying, feeling nervous, restlessness or loss of appetite. Suitably, the treatment may reduce the anxiety subscale score in the Children's Behaviour Checklist (CB CL) (also known as the Achenbach System of Empirically Based Assessment (ASEBA)), which is an instrument used to rate a child's problem behaviours and competencies (Achenbach 1991; Achenbach and Rescorla 2000).

Symptoms of depression that may be eliminated, reduced or ameliorated include finding it hard to concentrate, difficulty sleeping, irritability, crying, anxiety, restlessness, loss of appetite or feeling sad, tearful, empty or hopeless.

Suitably, the combination or composition according to the invention treats stress and/or a mood disorder by promoting relaxation in the infant, young child and/or child.

Suitably, the combination or composition according to the invention treats stress and/or a mood disorder by increasing production of gamma-aminobutyric acid (GABA) by the gut microbiota of the infant, young child and/or child. Many species of Lactobacillus and Bifidobacteria, including Bifidobacterium adolescentis, are able to synthesise and secrete the potent inhibitory neurotransmitter gamma-aminobutyric acid (GABA) which is known to be involved in reducing stress, anxiety and depression.

Accordingly, in another aspect, the invention provides a combination of galacto-oligosaccharides (GOS) and a mixture of human milk oligosaccharides (HMOs) for use in increasing production of gamma-aminobutyric acid (GABA) by the gut microbiota of an infant, young child and/or child, wherein the mixture of HMOs consists or consists essentially of 21-fucosyllactose (2′FL) and difucosyllactose (DFL) or of lacto-N-tetraose (LNT) and 6′ sialyllactose (6′SL).

In another aspect, the invention provides a nutritional composition comprising a combination of galacto-oligosaccharides (GOS) and a mixture of human milk oligosaccharides (HMOs) for use in increasing production of gamma-aminobutyric acid (GABA) by the gut microbiota of an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

Suitably, the biosynthesis of GABA by the gut microbiota of the infant, young child and/or child is increased from 1.1 to 20-fold, such as by 1.2 to 11-fold, preferably by 4-fold to 11-fold. Suitably, the biosynthesis of GABA by the gut microbiota of the infant, young child and/or child is increased by at least 1.1-fold, such as by 1.2-fold, 1.3-fold, 1.4-fold, 1.5-fold, 2-fold, 3-fold, 4-fold, 5-fold, 6-fold, 7-fold, 8-fold, 9-fold, 10-fold, 15-fold or 20-fold.

Suitably, the base-2 logarithm of the ratio between the value of GABA for the combination or composition according to the invention and the value of GABA for a corresponding milk control is increased by from 0.2 to 5, suitably from 0.25 to 3.8.

The amount of GABA secreted (i.e. GABA biosynthesis) by the gut microbiota of the infant, young child and/or child may be measured by methods known in the art. For example, the method disclosed herein may be used (see Example 2). Suitably, the amount of GABA in fresh fecal samples from an infant, young child or child fed the combination or nutritional composition according to the invention may be compared to samples from an infant, young child or child not exposed to a combination or nutritional composition according to the invention (Altaib et al., (2021), Microorganisms, 9:378).

The gut-brain axis consists of bidirectional communication between the central nervous system (CNS) and the enteric nervous system (ENS), linking emotional and cognitive centers of the brain with peripheral intestinal functions. The autonomic nervous system, hypothalamic-pituitary-adrenal (HPA) axis, and nerves within the gastrointestinal (GI) tract, all link the gut and the brain, allowing the brain to influence intestinal activities, including activity of functional immune effector cells; and the gut to influence mood, cognition, and mental health. Recent advances in research have described the importance of gut microbiota in influencing these interactions. This interaction between microbiota and gut-brain axis appears to be bidirectional, namely involving signaling from gut-microbiota to brain and from brain to gut-microbiota by means of neural, endocrine, immune, and humoral links (e.g. by means of the vagus nerve). In clinical practice, evidence of microbiota-GBA interactions comes from the association of dysbiosis with central nervous disorders (i.e. autism, anxiety-depressive behaviors) and functional gastrointestinal disorders.

Clinical, epidemiological, and immunological evidence suggest that the enteric microbiota influences the gut-brain axis (i.e., mental state, emotional regulation, neuromuscular function, and regulation of the HPA). For example, several mood disorders, such as anxiety disorder, depression, and autism spectrum disorders now have well-established links to functional GI disruptions and GI diseases (e.g., irritable bowel syndrome) often involve psychological comorbidities associated with alteration of the gut microbiome.

Without wishing to be bound by theory, by altering the microbiota to increase biosynthesis of GABA, amongst other impacts, the combination according to the invention may be capable of influencing neurotransmission in the paraventricular hypothalamus, the central nucleus of the amygdala, and the bed nucleus of the stria terminalis. All three of these regions are involved in the processing of emotions related to anxiety and mood.

Accordingly, in some embodiments, the combination or nutritional composition according to the invention maintains or restores the functionality of the bi-directional transmission pathways in the gut-brain axis to healthy levels in an infant, young child and/or child.

In another aspect, the invention provides a combination of galacto-oligosaccharides (GOS) and a mixture of human milk oligosaccharides (HMOs) for use in maintaining or restoring the functionality of the bi-directional transmission pathways in the gut-brain axis to healthy levels in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In another aspect, the invention provides a nutritional composition comprising a combination of galacto-oligosaccharides (GOS) and a mixture of human milk oligosaccharides (HMOs) for use in maintaining or restoring the functionality of the bi-directional transmission pathways in the gut-brain axis to healthy levels in an infant, young child and/or child, wherein the mixture of HMOs consists or consists essentially of 2′-fucosyllactose (2′FL) and difucosyllactose (DFL) or of lacto-N-tetraose (LNT) and 6′ sialyllactose (6′SL).

In a further aspect, the present invention provides a method for treating and/or preventing stress and/or a mood disorder in an infant, young child and/or child, the method comprising administering to an infant, young child and/or child a combination according to the invention or a nutritional composition according to the invention.

In a further aspect, the present invention provides a method for maintaining or restoring the functionality of the bi-directional transmission pathways in the gut-brain axis to healthy levels in an infant, young child and/or child, the method comprising administering to an infant, young child and/or child a combination according to the invention or a nutritional composition according to the invention.

In a further aspect, the present invention provides a method for increasing production of gamma-aminobutyric acid (GABA) by the gut microbiota of an infant, young child and/or child, the method comprising administering to an infant, young child and/or child a combination according to the invention or a nutritional composition according to the invention.

In some embodiments, the method for increasing production of gamma-aminobutyric acid (GABA) by the gut microbiota is non-therapeutic.

In some embodiments of the methods and uses of the invention, the combination or composition according to the invention is administered to the infant, young child and/or child in an effective amount.

In a further aspect, the present invention provides the use of a combination according to the invention or a nutritional composition according to the invention for treating and/or preventing stress and/or a mood disorder in an infant, young child and/or child. Suitably, the combination treats stress and/or a mood disorder by increasing production of gamma-aminobutyric acid (GABA) by the gut microbiota of the infant, young child and/or child.

In another aspect, the invention provides the use of a combination according to the invention or a nutritional composition according to the invention for maintaining or restoring the functionality of the bi-directional transmission pathways in the gut-brain axis to healthy levels in an infant, young child and/or child.

In another aspect, the invention provides the use of a combination according to the invention or a nutritional composition according to the invention for increasing production of gamma-aminobutyric acid (GABA) by the gut microbiota of an infant, young child and/or child.

In some embodiments, the use or method according to the invention is non-therapeutic. Suitably, the use or method is for increasing production of gamma-aminobutyric acid (GABA) by the gut microbiota of an infant, young child and/or child; for promoting relaxation in an infant, young child and/or child; for promoting calmness in an infant, young child and/or child; or for improving mood in an infant, young child and/or child.

In some embodiments of the methods and uses of the invention, the combination or composition according to the invention is used for the treatment of stress and/or a mood disorder.

In some embodiments of the methods and uses of the invention, the combination or composition according to the invention is used for the prevention of stress and/or a mood disorder.

In some embodiments of the methods and uses of the invention, the combination or composition according to the invention is used for the treatment and/or prevention of stress.

In some embodiments of the methods and uses of the invention, the combination or composition according to the invention is used for the treatment and/or prevention of a mood disorder.

### Target Population

In some embodiments of the methods and uses of the invention, the combination or composition administered to an infant.

In some embodiments of the methods and uses of the invention, the combination or composition is administered to a young child.

In some embodiments of the methods and uses of the invention, the combination or composition is administered to a child.

In one embodiment of the present invention, the infant or young children have an age ranging from 0 months to 36 months.

In another embodiment, the infant or young children have an age ranging from 0 months to 24 months, for example 18 months.

In a further embodiment, the infant has an age ranging from 0 months to 12 months, for example 9 or 6 months.

In some embodiments, the young child has an age ranging from one year to three years, for example 2 years.

In some embodiments, the child has an age ranging from three years to seven years. In some preferred embodiments, the child has an age ranging from three years to five years, for example four years.

Some specific populations of infants, young children and children are particularly in need of compositions able to treat and/or prevent stress and/or a mood disorder. Such infants, young children and children are for example preterm infants, low birth weight infant, and/or growth-retarded infants, young children or children. Indeed such subjects are often experiencing adverse medical conditions and require significantly more frequent medical intervention than term infants and infants having experienced normal development. Many of such medical interventions are unfortunately painful for the infant or young child, which is thus faced with repeated stress and/or a mood disorder. For such infants it is particularly advantageous to complement medical interventions with nutritional compositions capable of treating and/or preventing stress and/or a mood disorder.

The nutritional composition according to the invention is for use in infants, young children and/or children. The infants, young children and/or children may be born term or preterm. In a particular embodiment the nutritional composition of the invention is for use in infants, young children and/or children that were born preterm, having a low birth weight and/or born small for gestational age (SGA). In a particular embodiment the nutritional composition of the invention is for use in preterm infants, infants having a low birth weight and/or infants born small for gestational age (SGA).

The nutritional composition of the present invention may also be used in an infant, young child or child that was born by C-section or that was vaginally delivered.

In some embodiments the composition according to the invention can be for use before and/or during the weaning period. The nutritional composition can be administered (or given or fed) at an age and for a period that depends on the needs.

The nutritional composition can be for example given immediately after birth of the infants. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4, 6 or 12 months of birth of said infant. In some other embodiments, the nutritional composition of the invention is given few days (e.g. 1, 2, 3, 5, 10, 15, 20 . . . ), or few weeks (e.g. 1, 2, 3, 4, 5, 6, 7, 8, 9, 10 . . . ), or few months (e.g. 1, 2, 3, 4, 5, 6, 7, 8, 9, 10 . . . ) after birth. This may be especially the case when the infant is premature, but not necessarily.

In one embodiment the composition of the invention is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1^{st}, 2^{nd} or 4th month of life, during at least 1, 2, 4 or 6 months.

### Combinations

In one embodiment, the mixture of oligosaccharides consists or consists essentially of 2′FL and DFL.

In one embodiment of the invention, 2′FL is present in the combination or nutritional composition in an amount of 0.005-8 g/L of the combination or composition. In some embodiments, 2′FL may be in an amount of 0.01-3 g/L, such as 0.04-2 g/L or 0.05-1.5 g/L or 0.09-1.2 g/L. In a particular embodiment, 2′FL is in an amount of 1 g/L. In another particular embodiment, 2′FL is in an amount of 0.2 g/L.

2′FL can be present in the combination or nutritional composition in an amount of 0.004-6.8 g/100 g of the combination or composition on a dry weight basis, 2′FL may be present in an amount of 0.008-2.4 g/100 g, such as 0.03-1.6 g/100 g or 0.04-1.2 g/100 g or 0.07-1.0 g/100 g. In a particular embodiment, 2′FL is present in an amount of 0.8 g/100 g. In another particular embodiment, 2′FL is present in an amount of 0.16 g/100 g.

In another particular embodiment, 2′FL is in an amount of 5-500 g/L, 10 to 400 g/L, 40 to 300 g/L, 60-200 g/L, 80-180 g/L, 100-150 g/L or 110-130 g/L. In a particular embodiment, the 2′-FL is in an amount of 120 g/L. Such amounts are particularly adequate when the nutritional composition is in the form of a supplement or of a fortifier.

When the supplement or fortifier is in powder form 2′FL is preferably provided in the nutritional composition of the present invention in such an amount of 0.05-5 g, 0.1-4.5 g, 0.15-4 g, 0.2 to 3.5 g, 0.25 to 3, 0.3 to 2.5, 0.35 to 2, 0.4 to 1.5 g, 0.45-1 g, 0.5 to 0.75 g for example 0.6 g per serving.

In some embodiments, DFL is in an amount of 0.0005-0.5 g/L of the combination or composition, such as 0.001-0.3 g/L or 0.002-0.2 g/L or 0.01-0.25 g/L or 0.015-0.2 g/L or 0.025-0.19 g/L or 0.075-0.165 g/L of the combination or composition. In a particular embodiment, DFL is in an amount of 0.1 g/L. In another particular embodiment, DFL is in an amount of 0.025 or 0.026 g/L. In one embodiment DFL is in an amount of more than 0.01 g/L and optionally less than 0.1 g/L or more than 0.02 and less than 0.08 g/L. In one embodiment DFL is present in an amount of at least 0.01 g/L, at least 0.025, at least 0.026, at least 0.05, at least 0.07, at least 0.08, at least 0.1, at least 0.125, at least 0.15 at least 0.15 or at least 0.2 g/L.

In some embodiments, DFL is present in the combination or composition in a total amount of 0.0004-0.38 g/100 g of combination or composition on a dry weight basis. DFL may be in an amount of 0.0008-0.23 g/100 g, such as 0.0015-0.15 g/100 g, or 0.008-0.19 g/100 g or 0.012-0.15 g/100 g or 0.015-0.15 g/100 g or 0.019-0.15 g/100 g. In a particular embodiment, DFL is in an amount of 0.0075 or 0.078 g/100 g. In another particular embodiment, DFL is in an amount of 0.02 g/100 g. In a particular embodiment, DFL is in an amount of at least 0.001 g/100 g, at least 0.002 g/100 g, at least 0.005 g/100 g, at least 0.01 g/100 g, at least 0.02 g/100 g, at least 0.025 g/100 g, at least 0.04 g/100 g, at least 0.05 g/100 g, at least 0.075 g/100 g, at least 0.09 g/100 g, at least 0.1 g/100 g, at least 0.15 g/100 g, at least 0.2 g/100 g or at least 0.3 g/100 g.

In a particular embodiment, the DFL is provided in the combination or nutritional composition of the present invention in such an amount that normal consumption of the combination or nutritional composition would provide to the infant, young child, or child, consuming it a total daily dose of 0.003-3.9 g, preferably 0.006-3 g or 0.05-3 g or 0.1-3 g, for example 1.1-2.8 g per day.

In a particular embodiment, the DFL is provided in the combination or nutritional composition of the present invention in such an amount that one serving of the combination or nutritional composition would provide to the infant, young child, or child, consuming it a total dose of 0.003-3.9 g, preferably 0.006-3 g or 0.05-2.5 g or 0.1-2 g, for example 0.02 to 0.07 g per serving.

In a particular aspect of the invention, the combination or nutritional composition is an infant formula comprising an HMO mixture consisting or consisting essentially of 2′-FL and DFL wherein:
2′-FL is in an amount of 1-2 g/L, preferably 1.3 g/L, of the composition, and/or in an amount of 0.8-1.5 g/100 g, preferably 1 g/100 g of composition on a dry weight basis; and/or
DFL is in an amount of 0.1 to 0.3 g/L, preferably 0.2 g/L, of the composition and/or in an amount of 0.08-0.2 g/100 g, preferably 0.2 g/100 g of composition on a dry weight basis.

In another particular aspect of the invention, the combination or nutritional composition is a supplement or a fortifier and comprises an HMO mixture consisting or consisting essentially of 2′-FL and DFL wherein:
2′-FL is in an amount of 100-110 g/L, preferably 105 g/L, of the composition; and/or
DFL is in an amount of 10-20 g/L, preferably 15 g/L, of the composition.

In a particular aspect, 2′-FL and DFL are present in the HMO mixture in a ratio 2′-FL:DFL of from 5:1 to 14:1, such as 5:1 to 12:1, 5:1 to 10:1, 6:1 to 10:1 or 8:1 to 10:1. In particularly advantageous embodiments, this ratio is 9:1 or around 9:1.

2′-FL and DFL may be isolated by chromatography or filtration technology from a natural source such as animal milks. Alternatively, the HMO may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. 2′-FL and DFL formation can be initiated by acceptor substrates starting from any degree of polymerization (DP), from DP=1 onwards. Alternatively, 2′-FL and DFL may be produced by chemical synthesis from lactose and free fucose. 2′-FL and DFL are also available commercially, for example from Glycom A/S in Denmark or Jennewein GmBH in Germany. 2′FL may be synthesised as described for example in “Large-scale synthesis of H-antigen oligosaccharides by expressing Helicobacter pylori alpha1,2-fucosyltransferase in metabolically engineered Escherichia coli cells “(Drouillard S, Driguez H, Samain E. Angew Chem Int Ed Engl. 2006 Mar. 3; 45 (11): 1778-80) or be obtained from commercial sources.

In one embodiment of the present invention, the combination or nutritional composition comprises a mixture of HMOs consisting or consisting essentially of 2′FL and DFL. It is to be understood that when the expression “a mixture of HMOs consisting or consisting essentially of 2′FL and DFL” is used in the context of the present invention, such expression excludes the presence of HMOs different from 2′FL and DFL in the mix. In particular the mixture is devoid of N-acetylated oligosaccharides, sialylated oligosaccharides, fucosylated oligosaccharides other than 2′-FL and DFL and precursors of HMOs such as sialic acid or fucose. In some embodiments, the combination or nutritional composition is devoid of N-acetylated oligosaccharides, sialylated oligosaccharides, fucosylated oligosaccharides other than 2′-FL and DFL and precursors of HMOs such as sialic acid or fucose.

In one embodiment, the mixture of oligosaccharides consists or consists essentially of LNT and 6′SL.

In one embodiment of the invention, LNT is present in the combination or nutritional composition in an amount of 0.005-3 g/L of the composition. In some embodiments, LNT may be in an amount of 0.01-1.5 g/L of the composition, such as 0.04-1.2 g/L or 0.05-1 g/L or 0.09-0.8 g/L of the composition. In a particular embodiment, LNT is in an amount of 0.5 g/L of the composition. In another particular embodiment, LNT is in an amount of 0.1 g/L of the composition.

LNT can be present in the combination or nutritional composition in an amount of 0.004-2.3 g/100 g of composition on a dry weight basis, LNT may be present in an amount of 0.008-1.2 g/100 g of composition, such as 0.03-0.9 g/100 g or 0.04-0.8 g/100 g or 0.07-0.6 g/100 g of the composition. In a particular embodiment, LNT is present in an amount of 0.38 g/100 g of the composition. In another particular embodiment, LNT is present in an amount of 0.08 g/100 g of the composition.

In another embodiment of the invention the combination or nutritional composition may comprise from 0.005-5 g/L of 6′SL, or from 0.008-2.5 g/L, or from 0.01-1 g/L, or from 0.03-0.7 g/L, for example 0.04 or 0.5 g/L of LNT.

The nutritional composition according to the invention can contain 0.004-3.8 g of LNT per 100 g of composition on a dry weight basis, e.g. 0.006-1.9 g or 0.008-0.8 g or 0.023-0.5 g or 0.031-0.4 of LNT per 100 g of composition on a dry weight basis, for example 0.18 g or 0.04 g per 100 g of composition on a dry weight basis.

In a particular embodiment, the LNT is provided in the nutritional composition of the present invention in such an amount that normal consumption of the nutritional composition would provide to the infant or young child, respectively the child, consuming it a total daily dose of 0.003-3.9 g, preferably 0.006-2 g or 0.02-1.6 g or 0.03-1.3 g, for example 0.05-1 g per day.

LNT may be synthesised chemically by enzymatic transfer of saccharide units from donor moieties to acceptor moieties using glycosyltransferases as described for example in U.S. Pat. No. 5,288,637 and WO 96/10086.

6′SL formation can be initiated by acceptor substrates starting from any degree of polymerisation (DP), from DP=1 onwards. Alternatively, 6′SL may be produced by chemical synthesis from lactose and free N′-acetylneuraminic acid (sialic acid).

In one embodiment of the invention, 6′SL is present in the combination or nutritional composition in an amount of 0.005-5 g/L of the composition. In some embodiments, 6′SL may be in an amount of 0.08-2.5 g/L of the composition, such as 0.01-1 g/L or 0.03-0.07 g/L or 0.04-0.5 g/L of the composition. In a particular embodiment, 6′SL is in an amount of 0.24 g/L of the composition. In another particular embodiment, 6′SL is in an amount of 0.05 g/L of the composition.

6′SL can be present in the combination or nutritional composition in an amount of 0.004-3.8 g/100 g of composition on a dry weight basis, 6′SL may be present in an amount of 0.006-1.9 g/100 g of composition, such as 0.008-0.8 g/100 g or 0.23-0.5 g/100 g or 0.031-0.4 g/100 g of the composition. In a particular embodiment, 6′SL is present in an amount of 0.18 g/100 g of the composition. In another particular embodiment, 6′SL is present in an amount of 0.04 g/100 g of the composition.

In a particular aspect, the 6′SL and the LNT comprised in the composition or nutritional composition according to the invention are typically present in a ratio 6′SL:LNT of from 3:1 to 1:3, such as 2:1 to 1:2 or 2:1 to 1:1. In a particularly advantageous embodiment, this ratio is 2:1 or around 2.1 preferably this ratio is 1:1 or around 1:1.

In a particular aspect of the invention, the combination or nutritional composition comprises 6′SL and LNT wherein:
6′SL is in an amount of 0.005-5 g/L of the composition and/or in an amount of 0.004-3.8 g/100 g of composition on a dry weight basis; and/or
LNT is in an amount of 0.005-3 g/L of the composition and/or in an amount of 0.004-2.3 g/100 g of composition on a dry weight basis.

In a particular aspect of the invention, the combination or nutritional composition comprises 6′SL and LNT wherein:
6′SL is in an amount of 0.008-2.5 g/L of the composition and/or in an amount of 0.006-1.9 g/100 g of composition on a dry weight basis; and/or
LNT is in an amount of 0.01-1.5 g/L of the composition and/or in an amount of 0.008-1.2 g/100 g of composition on a dry weight basis.

In another particular embodiment the combination or nutritional composition of the present invention comprises 6′SL and LNT wherein:
6′SL is in an amount of 0.01-1 g/L of the composition and/or in an amount of 0.008-0.8 g/100 g of composition on a dry weight basis; and/or
LNT is in an amount of 0.04-1.2 g/L of the composition and/or in an amount of 0.03-0.9 g/100 g of composition on a dry weight basis.

In another particular embodiment the combination or nutritional composition of the present invention comprises 6′SL and LNT wherein:
6′SL is in an amount of 0.03-0.7 g/L of the composition and/or in a total amount of 0.023-0.5 g/100 g of composition on a dry weight basis; and/or
LNT is in an amount of 0.05-1 g/L of the composition and/or in an amount of 0.04-0.8 g/100 g of composition on a dry weight basis.

In another particular embodiment the combination or nutritional composition of the present invention comprises 6′SL and LNT wherein:
6′SL is in an amount of 0.04-0.5 g/L of the composition and/or in an amount of 0.031-0.4 g/100 g of composition on a dry weight basis; and/or
LNT is in an amount of 0.09-0.8 g/L of the composition and/or in an amount of 0.07-0.6 g/100 g of composition on a dry weight basis.

In a specific embodiment the combination or nutritional composition of the present invention comprises 6′SL and LNT wherein:
6′SL is in an amount of 0.24 or 0.05 g/L of the composition and/or in an amount of 0.18 or 0.04 g/100 g of composition on a dry weight basis; and/or
LNT is in an amount of 0.5 g/L or 0.1 g/L of the composition and/or in an amount of 0.38 g/100 g of composition or 0.08 g/100 g of composition on a dry weight basis.

In a particular embodiment, 6′SL is provided in the combination or nutritional composition of the present invention in such an amount that normal consumption of the nutritional composition or growing-up milk would provide to the infant, young child, or child, consuming it a total daily dose of 0.003 to 6.5 g, preferably 0.005-3.3 g or 0.006-1.3 g or 0.02-0.9 g, for example 0.024-0.7 g per day.

In a particular embodiment, the LNT is provided in the nutritional composition or growing-up milk of the present invention in such an amount that normal consumption of the nutritional composition or growing-up milk would provide to the infant, young child, or child, consuming it a total daily dose of 0.003-3.9 g, preferably 0.006-2 g or 0.02-1.6 g or 0.03-1.3 g, for example 0.05-1 g per day.

In one embodiment of the present invention, the combination or nutritional composition comprises a mixture of HMOs consisting or consisting essentially of LNT and 6′SL. It is to be understood that when the expression “a mixture of HMOs consisting or consisting essentially of LNT and 6′SL” is used in the context of the present invention, such expression excludes the presence of HMOs different from LNT and 6′SL in the mix. In particular the mixture is devoid of N-acetylated oligosaccharides, sialylated oligosaccharides, fucosylated oligosaccharides other than LNT and 6′SL and precursors of HMOs such as sialic acid or fucose. In some embodiments, the combination or nutritional composition is devoid of N-acetylated oligosaccharides, sialylated oligosaccharides, fucosylated oligosaccharides other than LNT and 6′SL and precursors of HMOs such as sialic acid or fucose.

In a preferred embodiment of the invention, the HMO mixture may be present in an amount of 0.15-15 g/L of the composition, such as 0.5-10 g/L or 0.75-7.5 g/L or 1-5 g/L or 1 to 2 g/L of the composition. In a particular embodiment, the HMO mixture is in an amount of 1.5 g/L of the composition. In a preferred embodiment, the HMO mixture is in an amount of 2 g/L of the composition. Such amounts are particularly adequate when the nutritional composition is in the form of a complete nutrition such as an infant formula, or in the case of a growing-up milk.

In case wherein the combination or nutritional composition is in powder form, the HMO mixture may preferably be present in an amount of 0.11-11 g/100 g of composition on a dry weight basis, such as 0.4-7.5 g/100 g or 0.6-6 g/L or 0.8-3.8 g/100 g or 0.8-1.5 g/100 g of composition on a dry weight basis. In a particular embodiment, the HMO mixture is in an amount of 1.1 g/100 of composition on a dry weight basis. Such amounts are particularly adequate when the combination or nutritional composition is in the form of a complete nutrition such as an infant formula, or in the case of a growing-up milk.

In another particular embodiment, the HMO mixture is in an amount of 5-500 g/L, 10 to 400 g/L, 40 to 300 g/L, 60-200 g/L, 80-180 g/L, 100-150 g/L or 110-130 g/L of the composition. In a particular embodiment, the HMO mixture is in an amount of 120 g/L. Such amounts are particularly adequate when the nutritional composition is in the form of a supplement or of a fortifier.

When the supplement or fortifier is in powder form the HMO mixture is preferably provided in the nutritional composition of the present invention in such an amount of 0.05-5 g, 0.1-4.5 g, 0.15-4 g, 0.2 to 3.5 g, 0.25 to 3, 0.3 to 2.5, 0.35 to 2, 0.4 to 1.5 g, 0.45-1 g, 0.5 to 0.75 g for example 0.6 g per serving.

In preferred embodiments of the invention, the nutritional composition is a synthetic nutritional composition.

In preferred embodiments of the invention, the HMO mixture of the combination or the nutritional composition consists of 2′-fucosyllactose (2′-FL) and difucosyllactose (DFL) or of lacto-N-tetraose (LNT) and 6′-sialyllactose (6′-SL).

In preferred embodiments of the invention, in the combination or in the (synthetic) nutritional composition, the HMOs provided by the HMO mixture consisting or consisting essentially of 2′-fucosyllactose (2′-FL) and difucosyllactose (DFL) or of lacto-N-tetraose (LNT) and 6′-sialyllactose (6′-SL) represent 95% to 100% by weight, preferably 97% to 100% by weight, more preferably of 99% to 100% by weight, most preferably 99.5% to 100% by weight, such as 99.9% to 100% by weight of the total amount of HMOs in the combination or the (synthetic) nutritional composition.

In preferred embodiments of the invention, the combination or the (synthetic) nutritional composition does not comprise any other HMO than the HMOs provided by the HMO mixture consisting of 2′-fucosyllactose (2′-FL) and difucosyllactose (DFL) or of lacto-N-tetraose (LNT) and 6′-sialyllactose (6′-SL).

In one embodiment, the HMO mixture is provided in the combination or nutritional composition of the present invention in such an amount that normal consumption of the nutritional composition would provide to the infant or young child, respectively the child, consuming it a total daily dose of 0.1 to 10 g, such as 0.2-9 g, 0.3-8 g, 0.4-7 g, 0.5-6 g, 0.6-5 g, 0.8-3 g, 0.9-2 g or 1 to 1.5 g per day.

When GOS, 2′FL and DFL are combined, all three oligosaccharides are considered responsible for the effect, e.g. for treating and/or preventing stress and/or a mood disorder.

When GOS, LNT and 6′SL are combined, all three oligosaccharides are considered responsible for the effect, e.g. for treating and/or preventing stress and/or a mood disorder.

The mixture of HMOs as described herein is used in combination with GOS.

In a particular embodiment, the GOS is provided in the combination or nutritional composition of the present invention in such an amount that one serving of the combination or nutritional composition would provide to the infant, young child, or child, consuming it a total dose of 0.003-3.9 g, preferably 0.006-3 g, 0.05-2.5 g, 0.1-2 g or 0.2-1.5 g, for example 0.36 to 1.22 g per serving. Suitably, the GOS is provided in the combination or nutritional composition of the present invention in such an amount that one serving of the combination or nutritional composition would provide to the infant, young child, or child, consuming it a total dose of 0.001, 0.005, 0.01, 0.005, 0.1, 0.5, 1, 1.5, 2, 2.5, 3, 3.5 or 4 g per serving, or any range formed thereby.

### Other Ingredients

The nutritional composition according to the present invention may also comprise other types of oligosaccharide(s) (i.e. other than human milk oligosaccharides mentioned above) and/or a fiber(s) and/or a precursor(s) thereof. The other oligosaccharide and/or fiber and/or precursor thereof may be selected from the list comprising HMOs, fructo-oligosaccharides (FOS), inulin, xylooligosaccharides (XOS), polydextrose and any combination thereof. They may be in an amount between 0 and 10% by weight of composition. In a particular embodiment, the nutritional composition or the growing-up milk can also contain at least one BMO (bovine milk oligosaccharide).

Additional HMOs which may be included in the combination or nutritional composition according to the present invention may be selected from the group consisting of 3-FL (3-fucosyllactose), Lacto-difucotetraose (LDFT)), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II, para-lacto-N-neohexaose (para-LNnH), LNnT (lacto-N-neotetraose), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose, lacto-N-decaose, 3-SL (3′ sialyllactose) and any combination thereof.

In some embodiments, the combination or nutritional composition according to the invention comprises at least one additional HMO. In other embodiments, the combination or nutritional composition according to the present invention is devoid of any further HMOs.

The nutritional composition of the present invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation KI2, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

The nutritional composition according to the invention may contain from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. There may be both viable probiotics and inactivated probiotics in some other embodiments. Probiotic components and metabolites can also be added.

The nutritional composition according to the invention can be for example an infant formula, a starter infant formula, a follow-on or follow-up formula, a growing-up milk, a baby food, an infant cereal composition, a fortifier such as a human milk fortifier, or a supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses. In such cases it is particularly useful to define the amount of 2′-FL and optionally other oligosaccharides in terms of daily dose to be administered to the infant or young child, such as described above.

When the nutritional composition is a supplement, it may comprise 2′-FL and no other additional nutrient on top of the excipients necessary to obtain a stable nutritional composition.

The nutritional composition of the present invention can be in solid (e.g. powder), liquid or gelatinous form. In a specific embodiment the nutritional composition is a supplement comprising 2-fucosylactose (2′-FL), wherein the supplement is in powder form and provided in a sachet, preferably a sachet with 0.1 to 20 g per sachet, for example 1 to 10 g of 2-fucosylactose (2′-FL) per sachet, or in the form of a syrup, preferably a syrup with a total solid concentration of 5 to 75 g/100 ml (5 to 75% (w/v)). When the supplement is in powder form, it may comprise a carrier. It is however preferred that the supplement is devoid of a carrier. When the supplement is in the form of a syrup, the HMOs are preferably dissolved or suspended in water acidified with citrate.

The nutritional composition according to the invention generally contains a protein source. The protein can be in an amount of from 1.6 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

The protein component can alternatively be replaced by a mixture or synthetic amino acid, for example for preterm or low birth weight infants.

In a particular embodiment the nutritional composition or the growing-up milk according to the invention is a hypoallergenic composition. In another particular embodiment the composition according to the invention is a hypoallergenic nutritional composition or growing-up milk.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, nucleotides, nucleosides, and the like.

The nutritional composition of the invention may also contain carotenoid(s). In some particular embodiments of the invention, the nutritional composition of the invention does not comprise any carotenoid.

The nutritional composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture. The fucosylated oligosaccharide(s) and the N-acetylated oligosaccharide(s) may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder.

The powder should have a moisture content of less than about 5% by weight. The fucosylated oligosaccharide(s) and the N-acetylated oligosaccharide(s) may also or alternatively be added at this stage by dry-mixing or by blending them in a syrup form of crystals, along with the probiotic strain(s) (if used), and the mixture is spray-dried or freeze-dried.

If a liquid composition is preferred, the homogenised mixture may be sterilised then aseptically filled into suitable containers or may be first filled into the containers and then retorted.

In another embodiment, the composition of the invention may be a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

### EXAMPLES

### Example 1

An example of the composition of an infant formula according to the present invention is given in the below table 1. This composition is given by way of illustration only.

TABLE 1
Composition of the infant formula of Example 1

| | | |
|---|---|---|
| Nutrients | per 100 kcal | per litre |

| Energy (kcal) | | | 100 | 670 |
|---|---|---|---|---|
| Protein (g) | | | 1.83 | 12.3 |
| Fat (g) | | | 5.3 | 35.7 |
| Linoleic acid (g) | | | 0.79 | 5.3 |
| α-Linolenic acid (mg) | | | 101 | 675 |
| Lactose (g) | | | 11.2 | 74.7 |
| Minerals (g) | | | 0.37 | 2.5 |
| Na (mg) | | | 23 | 150 |
| K (mg) | | | 89 | 590 |
| Cl (mg) | | | 64 | 430 |
| Ca (mg) | | | 62 | 410 |
| P (mg) | | | 31 | 210 |
| Mg (mg) | | | 7 | 50 |
| Mn (μg) | | | 8 | 50 |
| Se (μg) | | | 2 | 13 |
| Vitamin A (μg RE) | | | 105 | 700 |
| Vitamin D (μg) | | | 1.5 | 10 |
| Vitamin E (mg TE) | | | 0.8 | 5.4 |
| Vitamin K1 (μg) | | | 8 | 54 |
| Vitamin C (mg) | | | 10 | 67 |
| Vitamin B1 (mg) | | | 0.07 | 0.47 |
| Vitamin B2 (mg) | | | 0.15 | 1.0 |
| Niacin (mg) | | | 1 | 6.7 |
| Vitamin B6 (mg) | | | 0.075 | 0.50 |
| Folic acid (μg) | | | 9 | 60 |
| Pantothenic acid (mg) | | | 0.45 | 3 |
| Vitamin B12 (μg) | | | 0.3 | 2 |
| Biotin (μg) | | | 2.2 | 15 |
| Choline (mg) | | | 10 | 67 |
| Fe (mg) | | | 1.2 | 8 |
| I (μg) | | | 15 | 100 |
| Cu (mg) | | | 0.06 | 0.4 |
| Zn (mg) | | | 0.75 | 5 |
| | Oligosaccharides | 2′-FL (g) | 0.2 | 1.3 |
| | | DiFL (g) | 0.003 | 0.2 |
| | | GOS (g) | 0.64 | 4.3 |

### Example 2

### Methods

### In Vitro Digestion and Fermentation

A milk matrix (a standard milk follow-up formula) was subjected to oral, gastric and small intestinal digestion procedures according to the standardized INFOGEST 2.0 method (Brodkorb et al., Nature Protocols, 14:991-1014 (2019)). Then in vitro fermentation was achieved for 24 h in reactor containing:
10% of the digested milk matrix
80% of fermentation medium containing microbiota derived from fresh fecal samples, recovered from 3 healthy toddlers aged 3.2-3.3 years-old
10% of the test ingredients, consisting of
   Buffer only
   Galacto-oligosaccharides at the final concentration of 2 g/L
   Blend of 2′FL and DFL (ratio 9:1), at a final concentration of 2 g/L
   Blend of LNT and 6′SL (ratio 2:1), at a final concentration of 2 g/L
   A combination of (i) GOS and (ii) the blend of 2′FL and DFL (ratio 9:1), both at a final concentration of 2 g/L
   A combination of (i) GOS and (ii) the blend of LNT and 6′SL (ratio 2:1), both at a final concentration of 2 g/L

Each combination was tested in triplicate. After 24 hours, fermentation medium was collected for metabolomics analysis.

### Metabolomics

The untargeted LC-MS/MS was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Doneanu et al. (Waters (2011) application note https://www.waters.com/webassets/cms/library/docs/720004042en.pdf). Peak areas were extracted using Compound Discoverer 3.1 (Thermo Scientific). In addition to the automatic compound extraction by Compound Discoverer 3.1, a manual extraction of compounds included in an in-house library was performed using Skyline 21.1 (MacCoss Lab Software).

For each blend B, we calculate log 2 (GABA (B)/GABA (Milk)) that is, the base-2 logarithm of the ratio between the value of GABA for the blend and the value of GABA for milk.

### Statistics

Mixed linear models with random intercepts were used to assess the effect of the blends on the level of each metabolite.

The goodness of fit was assessed using the marginal and the conditional R2. The marginal R2 (marginal coefficient of determination) represents the variance explained by the fixed effects while the conditional R2 is interpreted as a variance explained by the entire model, including both fixed and random effects. In addition, we inspected the standardized residuals of each model to check whether outliers in the data were potentially of concern. The ratio of the between-cluster variance to the total variance is called the Intraclass Correlation (ICC) was generally between 0.6 and 0.8. This can also be interpreted as the correlation among observations within the same cluster (donor).

P-values, approximated with the Kenward-Roger method, were used to determine which blends were significantly associated with the outcome.

### Results

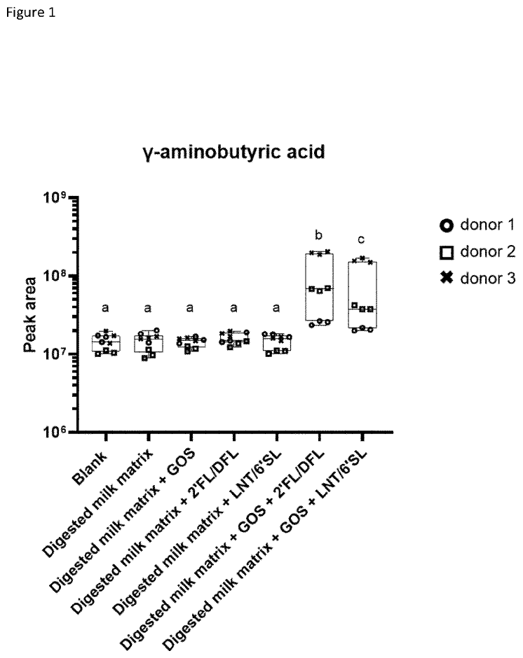

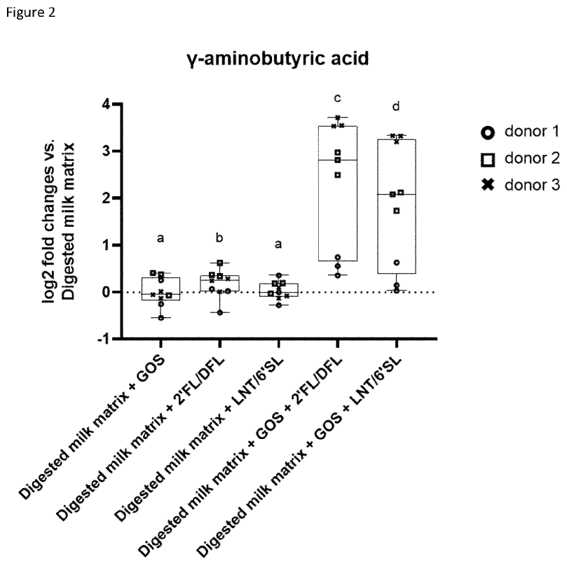

Exposure of microbiome to digested milk matrix, in presence of either GOS, a blend of 2′FL and DFL or a blend of LNT and 6′SL, did not trigger any effect on GABA abundance (FIG. 1). Conversely, the presence of a blend comprising GOS plus HMOs (2′FL/DFL or LNT/6′SL) strongly increased the synthesis of GABA by the microbiota compared to single ingredients (FIG. 2). The amount of GABA secreted was higher upon exposure with GOS+2′FL/DFL than GOS+LNT/6′SL.

1. A method for use in treating and/or preventing stress and/or a mood disorder in an infant, young child and/or child, comprising administering a combination of galacto-oligosaccharides (GOS) and a mixture of human milk oligosaccharides (HMOs) to the infant or child, wherein the mixture of HMOs consists or consists essentially of 2′-fucosyllactose (2′FL) and difucosyllactose (DFL) or of lacto-N-tetraose (LNT) and 6′ sialyllactose (6′SL).

2. (canceled)

3. The method of claim 1, wherein the combination restores the functionality of the bi-directional transmission pathways in the gut-brain axis to healthy levels.

4. The method according to claim 1, wherein the combination increases the production of gamma-aminobutyric acid (GABA) by the gut microbiota of the infant, young child and/or child and thereby treats stress and/or a mood disorder in the infant, young child and/or child.

5. The method according to claim 1, wherein the mood disorder is anxiety or depression.

6. The method according to claim 1, wherein the GOS are present in an amount of 2.4 g/L or 1.1 to 2.8 g/100 g of composition on a dry weight basis.

7. The method according to claim 1, wherein the mixture of HMOs consists or consists essentially of 2′FL and DFL.

8. (canceled)

9. The method according to claim 1, wherein the mixture of HMOs consists or consists essentially of LNT and 6′SL.

10. (canceled)

11. The method according to claim 1, wherein:
i) a) 2′FL is present in an amount of 0.005-8 g/L or 0.004-6.8 g/100 g of composition on a dry weight basis of composition on a dry weight basis; and
b) DFL is present in an amount of 0.1 to 0.3 g/L or 0.08-0.2 g/100 g of composition on a dry weight basis.

12. (canceled)

13. The method according to claim 1, wherein the combination further comprises at least one probiotic in an amount of from 103 to 1012 cfu/g of said combination (dry weight).

14. (canceled)

15. The method according to claim 1, wherein infant is a preterm infant, an infant having a low birth weight and/or an infant born small for gestational age (SGA).

16. (canceled)

17. A method for maintaining or restoring the functionality of the bi-directional transmission pathways in the gut-brain axis to healthy levels in an infant, young child and/or child, the method comprising administering to an infant, young child and/or child an effective amount of a combination of galacto-oligosaccharides (GOS) and a mixture of human milk oligosaccharides (HMOs) to the infant or child wherein the mixture of HMOs consists or consists essentially of 2′-fucosyllactose (2′FL) and difucosyllactose (DFL) or of lacto-N-tetraose (LNT) and 6′ sialyllactose (6′SL).

18. A method for increasing production of gamma-aminobutyric acid (GABA) by the gut microbiota of an infant, young child and/or child, the method comprising administering to an infant, young child and/or child an effective amount of combination of galacto-oligosaccharides (GOS) and a mixture of human milk oligosaccharides (HMOs) to the infant or child wherein the mixture of HMOs consists or consists essentially of 2′-fucosyllactose (2′FL) and difucosyllactose (DFL) or of lacto-N-tetraose (LNT) and 6′ sialyllactose (6′SL).

19. The method according to claim 18, wherein the method is non-therapeutic.

20.-24. (canceled)
